(19) United States
(12) Patent Application Publication
(10) Pub. No.: US 2020/0405545 A1
(43) Pub. Date: Dec. 31, 2020

(54) Q-TIPS AND A METHOD FOR PROVIDING THEM

(71) Applicant: Moshe Yoram Sharon, Rosh Pinna (IL)

(72) Inventor: Moshe Yoram Sharon, Rosh Pinna (IL)

(21) Appl. No.: 16/553,204

(22) Filed: Aug. 28, 2019

(30) Foreign Application Priority Data
Jun. 25, 2019 (IL) 267654

(51) Int. Cl. A61F 13/38; A46B 1/00

(52) U.S. Cl. CPC A61F 13/38; A46B 2200/1006; A46B 1/00

(57) ABSTRACT

It is disclosed a Q-tip which includes rolled up soft paper sheets, divided at one or two ends to a plurality of strips such as to form one or two respective brushes, and sticker or wire for gripping the rolled up soft paper sheets at a certain distance from the one or more brushes. The Q-tip includes one to four rolled up soft paper sheets. A method for providing a Q-tip may include providing soft paper sheets, dividing the soft paper sheets at two opposite ends to a plurality of strips, rolling up the soft paper sheets such as to form two brushes and a cylindrical part having an axis in parallel to the dividing direction, and gripping the soft paper sheets at the cylindrical part. The strips maybe of a substantially same width in the range of 7-8 mm, and a length in the range of 20-30 mm. A length of a soft paper sheet in the dividing direction is in the range of 95-105 mm and its width is in the range of 85-95 mm. Each brush may have 30-75 strips.

### CROSS REFERENCE

The application claims the priority rights of Israeli patent application No. 267654 entitled “COSMOS PURE” filed in Jun. 25, 2019.

### BACKGROUND OF THE INVENTION

### Field of the Invention

The invention is in the field of Q-tips used to clean the ears of children and adults.

### Background

Q-tips or ear toothpicks are used to clean and/or dry the ears of children and adults. The common Q-tip is made of a plastic or wood stick having two cotton swabs or cotton wool bundles attached to the respective two ends. The common q-tips have disadvantages both in operation and after operation. In operation, the stick hard material may cause harm to the soft tissues of the ear canal, as shown by medical research published in the scientific literature. Thus, using soft stick material may prevent such a harm.

After operation, the common Q-tips fail to disintegrate over time and consequently accumulate at landfills for many years. Thus, Q-tips which disintegrate rapidly are advantageous over the conventional Q-tips.

### BRIEF SUMMARY OF THE INVENTION

It is disclosed, according to preferred embodiments of the present disclosure, a Q-tip which includes rolled up soft paper sheets, divided at one or two ends to a plurality of strips such as to form one or two respective brushes. The Q-tip also includes means for gripping the rolled up soft paper sheets at a certain distance from the one or more brushes.

In a preferred embodiment, the Q-tip includes one rolled up soft paper sheet.

In a preferred embodiment, the Q-tip includes two, three, or four rolled up soft paper sheets.

In a preferred embodiment, the Q-tip includes two opposite brushes.

In a preferred embodiment, the Q-tip is tightened by a sticker.

In a preferred embodiment, the Q-tip is tightened by a wire.

It is disclosed, according to preferred embodiments of the present disclosure, a method for providing a Q-tip which includes a step of providing soft paper sheets, a step of dividing the soft paper sheets at two opposite ends to a plurality of strips in a dividing direction, a step of rolling up the soft paper sheets such as to form one or two brushes and a cylindrical part having an axis in parallel to the dividing direction, and a step of gripping the soft paper sheets at the cylindrical part.

In one preferred embodiment, a length of a soft paper sheet in the dividing direction is in the range of 95-105 mm, and its width transverse to the dividing direction is in the range of 85-95 mm.

In one preferred embodiment, most of the strips are of a substantially same width in the range of 3-9 mm, and having a length in the range of 20-30 mm. Preferably, each brush has 30-75 strips.

In one preferred embodiment, a diameter of the cylindrical part is in the range of 3-4 mm.

### BRIEF DESCRIPTION OF THE DRAWINGS

The subject matter regarded as the invention is particularly pointed out and distinctly claimed in the concluding portion of the specification. The invention, however, both as to article features and method of production, together with features and advantages thereof, may best be understood by reference to the following detailed description when read with the accompanied drawings in which:

FIG. 1 depicts a soft paper sheet for providing a Q-tip.

FIG. 2 shows a plurality of soft paper sheets for providing a single Q-tip.

FIG. 3 exhibits a cut soft paper sheet in a folding process.

FIG. 4a illustrates a Q-tip ready for use in a side view.

FIG. 4b is an enlarged upper view of the brush.

FIG. 5 is a flow chart of a method for providing Q-tips.

FIG. 6 depicts a Q-tip in use.

### DETAILED DESCRIPTION OF THE INVENTION

The present invention will now be described in terms of specific example embodiments. It is to be understood that the invention is not limited to the example embodiments disclosed. It should also be understood that not every feature of the methods and systems handling the described game is necessary to implement the invention as claimed in any particular one of the appended claims. Various elements and features of the invented article are described to fully enable the invention. It should also be understood that throughout this disclosure, where a method is shown or described, the steps of the method may be performed in any order or simultaneously, unless it is clear from the context that one step depends on another being performed first.

Before explaining several embodiments of the invention in detail, it is to be understood that the invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein is for the purpose of description and should not be regarded as limiting.

Unless otherwise defined, all technical and scientific terms used herein have the same meaning as commonly understood by one of ordinary skill in the art to which this invention belongs. The systems, methods, and examples provided herein are illustrative only and not intended to be limiting.

In the description and claims of the present application, each of the verbs “comprise”, “include” and “have”, and conjugates thereof, are used to indicate that the object or objects of the verb are not necessarily a complete listing of members, components, elements or parts of the subject or subjects of the verb.

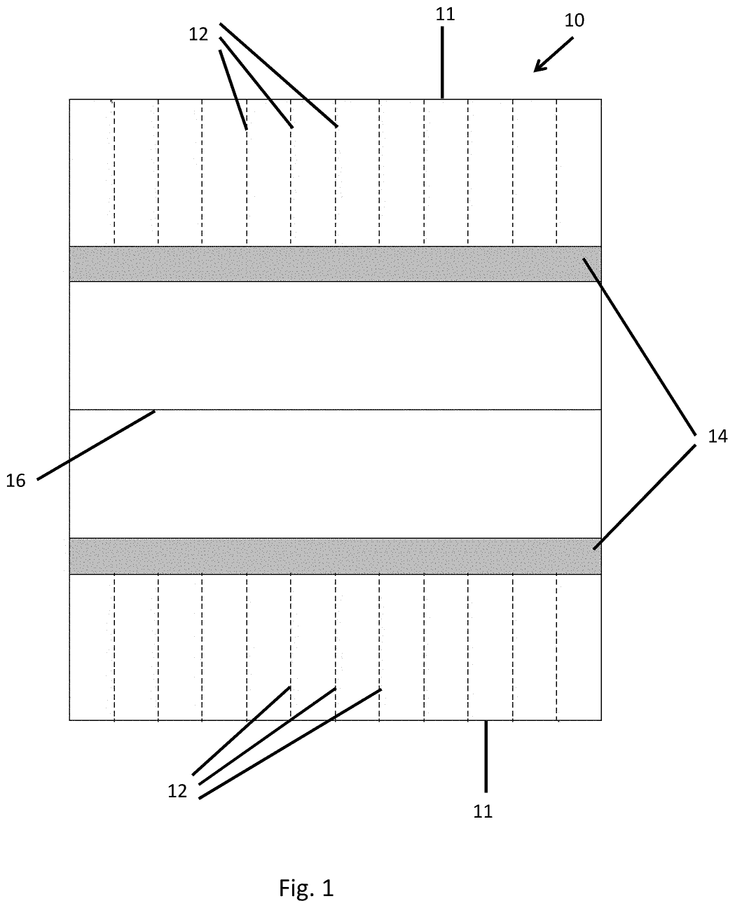

In the following, the description of the present invention begins with a method for producing a Q-tip, or an ear toothpick. The production starts with providing a soft paper sheet 10, as shown in FIG. 1. The soft paper sheet 10 is made of thin absorbing soft paper like the paper used commercially for tissues. The sheet is rectangular at a length in the range of 95-105 mm, and a width in the range of 85-95 mm.

Two end parts 11 in the long direction of the sheet, hereafter the dividing direction, are designated by two series of cut lines. A line may be a written or a cut dash line. Alternatively, the lines are virtual lines without any physical presence. A cutting device, as detailed below, is aligned such as to cut along the virtual lines. The lines designate strips 30 of length in the range of 20-30 mm and width in the range of 3-9 mm. Preferably, most of the strips 30 are of a substantially same width.

In one preferred embodiment, the soft paper sheet 10 includes elongated portions 14 disposed transversely to the dividing direction at the border of the end parts 11. Portions 14 are designated as portions excluded from being covered by sticker 46. Alternatively, portions 14 have physical feature that makes them distinct over the rest of page 10, like extra hardness, or different color.

A central line 16 may be designated on the sheet 10, as well.

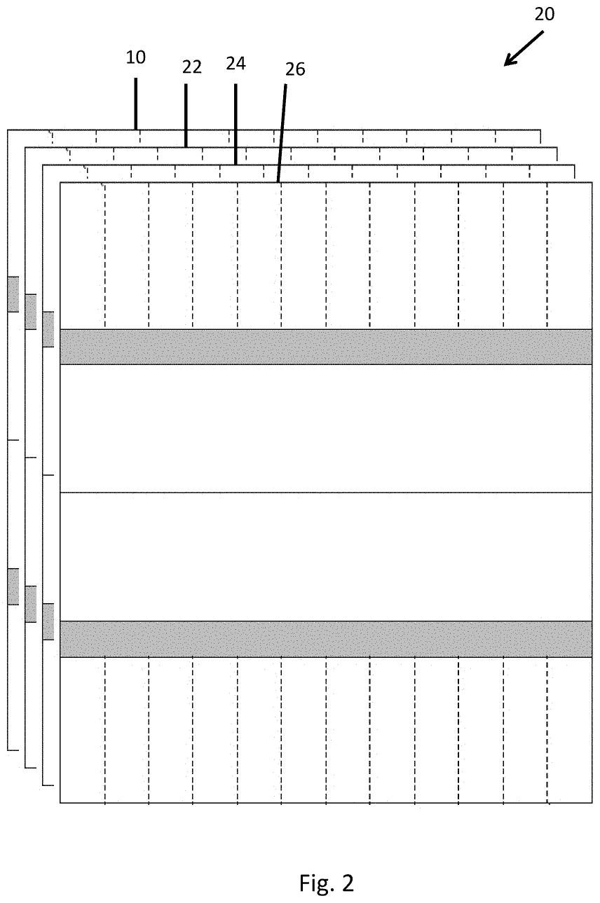

Rather than using a single sheet 10 for a single Q-tip, several sheets 10,22,24,26 may be used in a stack 20 for a single Q-tip as shown in FIG. 2.

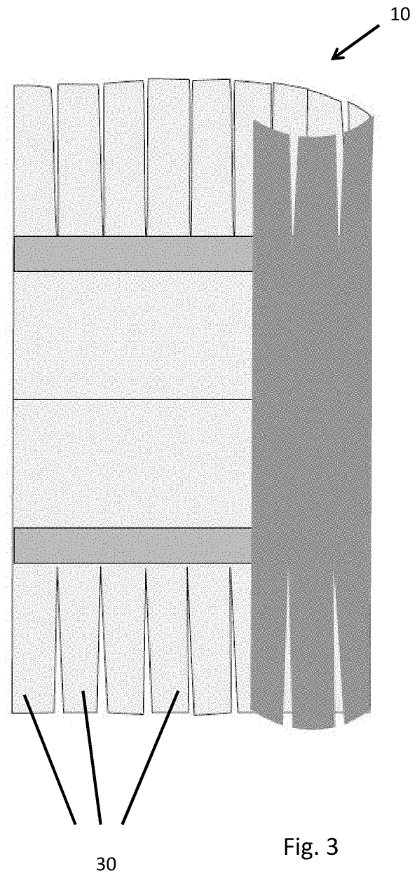

After having the soft paper sheet, one uses scissors to divide the sheet ends 11 to a plurality of strips 30, as shown in FIG. 3. Alternatively, an automatic production line which includes a knife, or a series of knives, is applied to divide the sheet ends 11 to a plurality of strips 30.

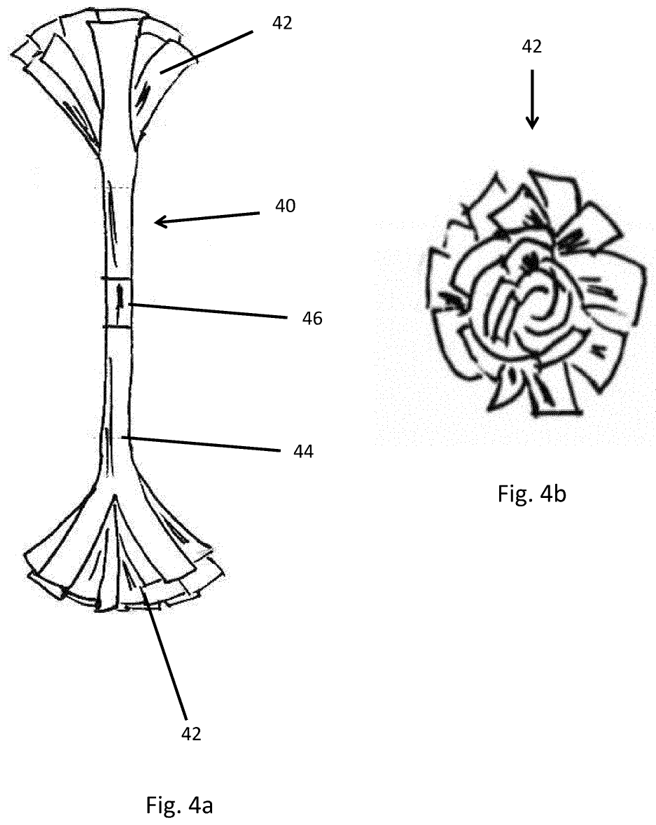

In the next production step, the sheet 10 or the sheet stack 20 is rolled up as depicted in FIG. 3 to form two brushes 42 and a cylindrical part 44 having an axis in parallel to the dividing direction, as shown in FIGS. 4a and 4b. Each brush has 30-75 strips. A diameter of the cylindrical part is in the range of 3-4 mm.

Finally, the cylindrical part 44 is gripped at the center by a sticker 46, and the resulted Q-tip 40 is ready for use. Sticker 46 prevents undesired unfolding of the folded sheet or sheet stack both in the longitudinal (dividing) direction and in the transverse direction. It also provides some hardness to the Q-tip such a to prevent undesired shrinking.

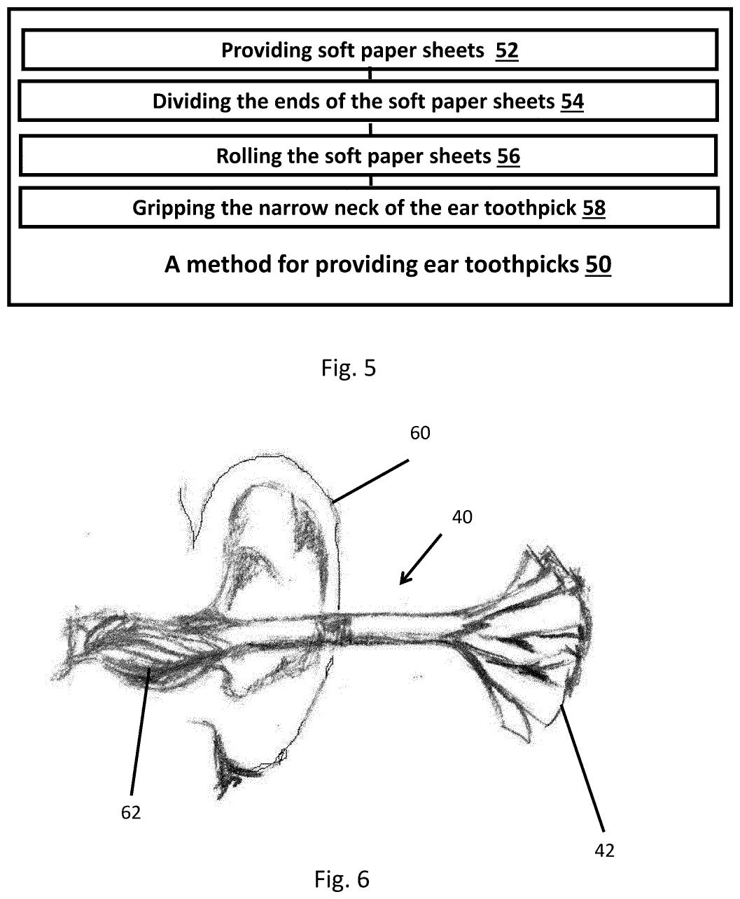

The method for providing Q-tips is reproduced in the flow chart of FIG. 5. The method 50 includes a step 52 of providing soft paper sheets 10, and a step 54 of dividing the soft paper sheets 10 at two opposite ends 11 into a plurality of strips 30 in a dividing direction. The method 50 also includes a step 56 of rolling up the soft paper sheets 10 such as to form two brushes 42 and a cylindrical part 44 having an axis in parallel to the dividing direction, and a step 58 of gripping the soft paper sheets 10 at the cylindrical part 44.

FIG. 6 illustrates the operation of the Q-tip 40. A user may insert the brush 62 into the ear 60 and may rotate the Q-tip 40 for cleaning the ear or for drying it after a shower, a bath or a swimming pool. The soft paper absorbs water or ear wax gently and after removal the ear canal is dry and clean. Once the Q-tip arrive a landfill, it disintegrates rather rapidly and causes no negative environmental effects.

The Q-tip 40 includes rolled up soft paper sheets 10, which have each two divided ends 11, such as to form a plurality of strips 30 in two respective brushes 42. The Q-tip 40 also includes means 46 for gripping the rolled up soft paper sheets 10 at a certain distance from the brushes 42.

In a preferred embodiment, the Q-tip 40 is gripped or tightened by a sticker. For environment conservation, the sticker may be made from paper only, not a plastic sticker. Alternatively, the Q-tip 40 is tightened by a wire 46.

The Q-tip of the current invention may not include any plastic part, unlike the more common Q-tips whereas two bundles of cotton wool are positioned at the ends of a plastic stick. Thus, the material of the Q-tip 40 disintegrates naturally and disappears in landfills eventually. Thus, it does not cause a damage to the environment as plastic Q-tip causes.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. In particular, the present invention is not limited in any way by the described examples.

1. A Q-tip comprising:
a. at least one rolled up soft paper sheet;
b. said at least one rolled up soft paper sheet being divided at one or two ends to a plurality of strips such as to form one or two respective brushes; and
c. means for gripping said at least one rolled up soft paper sheet at a certain distance from the one or more brushes.

2. The Q-tip of claim 1 wherein said Q-tip comprises one rolled up soft paper sheet.

3. The Q-tip of claim 1 wherein said Q-tip comprises two, three, or four rolled up soft paper sheets.

4. The Q-tip of claim 1 wherein said Q-tip comprises two opposite brushes.

5. The Q-tip of claim 1 wherein said Q-tip is tightened by a sticker.

6. The Q-tip of claim 1 wherein said Q-tip is tightened by a wire.

7. A method for providing a Q-tip including:
a. providing at least one soft paper sheet;
b. dividing said at least one soft paper sheet at one end or at two opposite ends to a plurality of strips in a dividing direction;
c. rolling up said at least one soft paper sheet such as to form one or two brushes, and a part of substantially cylindrical shape having an axis in parallel to the dividing direction; and
d. gripping the at least one rolled up soft paper sheet at the cylindrical part.

8. The method of claim 7 wherein one soft paper sheet is provided.

9. The method of claim 7 wherein two, three, or four soft paper sheets are provided.

10. The method of claim 7 wherein the provided Q-tip includes two opposite brushes.

11. The method of claim 7 wherein said cylindrical part is gripped by a sticker.

12. The method of claim 7 wherein said cylindrical part is gripped by a wire.

13. The method of claim 7 wherein most of the strips are of a substantially same width.

14. The method of claim 7 wherein said at least one soft paper sheet includes a hardened elongated portion disposed transversely to the dividing direction.

15. The method of claim 7 wherein a length of said at least one soft paper sheet in said dividing direction is in the range of 95-105 mm.

16. The method of claim 7 wherein a width of said at least one soft paper sheet transverse to said dividing direction is in the range of 85-95 mm.

17. The method of claim 7 wherein a width of the strips is in the range of 3-9 mm.

18. The method of claim 7 wherein a length of the strips is in the range of 20-30 mm.

19. The method of claim 7 wherein each brush has 30-75 strips.

20. The method of claim 7 wherein a diameter of the cylindrical part is in the range of 3-4 mm.
